(19) United States
(12) Patent Application Publication
(10) Pub. No.: US 2025/0336482 A1
(43) Pub. Date: Oct. 30, 2025

(54) TRAINING METHOD AND TRAINING APPARATUS FOR MACHINE LEARNING FORCE FIELDS MODEL

(71) Applicant: SAMSUNG ELECTRONICS CO., LTD., SUWON-SI (KR)

(72) Inventors: Yujie ZENG, Xi’an (CN); Vasyltsov IHOR, Suwon-si (KR); Wenlong HE, Xi’an (CN); Jiaxin WEI, Xi’an (CN); Lin CHEN, Xi’an (CN); Ying ZHANG, Xi’an (CN); Yuehua DAI, Xi’an (CN); Zihao LIU, Xi’an (CN)

(21) Appl. No.: 19/075,239

(22) Filed: Mar. 10, 2025

(30) Foreign Application Priority Data
Apr. 30, 2024 (CN) 202410544264.7
Oct. 31, 2024 (KR) 10-2024-0152376

(51) Int. Cl. G16C 10/00; G06F 30/27; G16C 20/70

(52) U.S. Cl. CPC G16C 10/00; G06F 30/27; G16C 20/70

(57) ABSTRACT

A method for training a machine learning force fields (MLFF) model, the method including obtaining, using the MLFF model, edge features corresponding to a training sample, wherein the training sample includes data related to a plurality of atoms, and the edge features represent relationship between edges of each atom among the plurality of atoms, computing a correlation loss corresponding to the edge features, wherein the correlation loss represents correlation between edge features corresponding to the plurality of atoms, updating parameters of the MLFF model based on the correlation loss to obtain a trained MLFF model, and generating, using the trained MLFF model, a molecular dynamics (MD) simulation based on an input sample.

### CROSS-REFERENCE TO RELATED APPLICATIONS

This U.S. non-provisional patent application claims priority under 35 U.S.C. § 119 to Chinese Patent Application No. 202410544264.7 filed on Apr. 30, 2024, in the China National Intellectual Property Administration, and Korean Patent Application No. 10-2024-0152376 filed on Oct. 31, 2024, in the Korean Intellectual Property Office, the entire disclosures of which are incorporated by reference herein in their entireties.

### BACKGROUND

### 1. Field of the Invention

Embodiments of the present disclosure relate to the field of artificial intelligence technology and, more particularly, to a training method and training apparatus for a machine learning force fields model.

### 2. Discussion of the Related Art

Molecular dynamics (MD) simulation is a technique widely used in the study of materials and biological systems. MD simulation provides a theoretical framework for simulating motions of interacting particle systems. Machine learning force fields (MLFF) describe MD force fields based on the positions of particles. In some cases, an MLFF model may be trained using training data that includes position information of the particles, the characteristics of the particles, and spatial features. The MLFF model may predict the energy of each particle and the force received by the particle. In addition, a molecular dynamics simulation tool, such as a large-scale atomic/molecular massively parallel simulator (LAMMPS), may compute updated positions of particles after each timestep based on the predicted force.

In MD simulation, prediction stability is an important objective. For example, if a trained MLFF model fails to predict data distribution using insufficient sampling during long-term simulation, then the trained MLFF model may generate unstable prediction results. The simulation may reach a non-physical status due to the failed prediction, resulting in a collapse of an MD simulation system.

### SUMMARY

Embodiments of the present disclosure provide a method, apparatus, non-transitory computer readable medium, and system for training a machine learning force fields (MLFF) model including obtaining, using the MLFF model, edge features corresponding to a training sample, wherein the training sample includes data related to a plurality of atoms, and the edge features represent relationship between edges of each atom among the plurality of atoms, computing a correlation loss corresponding to the edge features, wherein the correlation loss represents correlation between edge features corresponding to the plurality of atoms, updating parameters of the MLFF model based on the correlation loss to obtain a trained MLFF model, and generating, using the trained MLFF model, a molecular dynamics (MD) simulation based on an input sample.

Embodiments of the present disclosure provide an apparatus for training an MLFF model, the training apparatus at least one processor, at least one memory storing instructions executable by the at least one processor, an edge feature module comprising parameters stored in the at least one memory and configured to obtain, using the MLFF model, edge features corresponding to a training sample, wherein the training sample includes data related to a plurality of atoms, and the edge features represent relationship between edges of each atom among the plurality of atoms, a correlation loss module comprising parameters stored in the at least one memory and configured to perform compute a correlation loss corresponding to the edge features, wherein the correlation loss represents correlation between edge features corresponding to the plurality of atoms, and an updating module comprising parameters stored in the at least one memory and configured to update parameters of the MLFF model based on the correlation loss to obtain a trained MLFF model.

### BRIEF DESCRIPTION OF THE DRAWINGS

FIG. 1 is a diagram illustrating a process of performing molecular dynamics (MD) simulation using a machine learning force fields (MLFF) model according to an embodiment of the present disclosure.

FIG. 2 is a diagram illustrating an atomic structure of hafnium oxide (HfO) before simulation according to an embodiment of the present disclosure.

FIG. 3 is a diagram illustrating an atomic structure of hafnium oxide (HfO) after simulation according to an embodiment of the present disclosure.

FIG. 4 is a flowchart illustrating a method for training an MLFF model according to an embodiment of the present disclosure.

FIG. 5 is a diagram illustrating a plurality of layers of an MLFF model according to an embodiment of the present disclosure.

FIG. 6 is a diagram illustrating a method for training an MLFF model according to an embodiment of the present disclosure.

FIG. 7 is a block diagram of a training apparatus of an MLFF model according to an embodiment of the present disclosure.

FIG. 8 is a block diagram of an electronic device according to an embodiment of the present disclosure.

### DETAILED DESCRIPTION

The following structural or functional description is provided merely as an example and various alterations and modifications may be made to the embodiments. Here, the embodiments are not construed as limited to the disclosure and should be understood to include all changes, equivalents, and replacements within the idea and the technical scope of the disclosure.

Although terms of “first,” “second,” and the like are used to explain various components, the components are not limited to such terms. These terms are used to distinguish one component from another component. For example, a first component may be referred to as a second component, or similarly, the second component may be referred to as the first component within the scope of the present disclosure.

When it is mentioned that one component is “connected” or “accessed” to another component, it may be understood that the one component is directly connected or accessed to another component or an additional component may be interposed between the two components.

The singular forms “a”, “an”, and “the” are intended to include the plural forms as well, unless the context clearly indicates otherwise. As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical or scientific terms have the same meaning as commonly understood by one of ordinary skill in the art to which examples belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments are described in detail with reference to the accompanying drawings. When describing the embodiments with reference to the accompanying drawings, like reference numerals refer to like components and a repeated description related thereto may be omitted.

Molecular dynamics (MD) simulation is a technique widely used in the study of materials and biological systems. MD simulation provides a theoretical framework for simulating motions of interacting particle systems. MD simulation may be used in various application fields, such as new material design and optimization in the field of materials, drug design in the field of biology, catalyst research in the field of chemistry, etc.

Machine learning-based force fields models, such as spectral neighbor analysis potential (SNAP) or a rapid atomistic neural network (RANN), or may be referred to as machine learning force fields (MLFF) model, may generate results having higher accuracy than traditional force fields models. For example, an MLFF model based on a graph neural network (GNN) may be used in MD simulation. The accuracy of the MLFF model may be evaluated through the force mean absolute error (MAE) of each particle (or atom) and the energy MAE of each particle (or atom).

Conventional systems often present issues such as simulation instability and lack of robustness. For example, simulation instability may result in non-physical status, where atoms behave unrealistically, such as leaving the simulation container or clustering unnaturally. Additionally, conventional systems cannot dynamically adjust training, which further results in simulation instability and poor accuracy.

Embodiments of the present inventive concept provide a system and a method for training the MLFF model by using a correlation loss. For example, the system generates a correlation loss that minimizes the correlation between edge features in the MLFF model. Edge features represent relationships between atoms in the simulated system. By minimizing the correlation loss, the system improves the simulation stability. Additionally, by using the correlation loss, the system can generate a more accurate MD simulation given an input sample.

In some aspects, the correlation loss coefficient improves the simulation stability of the MLFF model by dynamically updating the weights of the correlation loss during training. For example, during the early training stage, higher weights are used to minimize feature correlation to increase simulation stability, while lower weights in later epochs (or later training stage) focus on increasing the accuracy in force and energy predictions. Accordingly, the training method of the present disclosure improves the simulation stability of the MLFF model while preventing a decrease in the accuracy of the MLFF model by using the dynamic weights during the training stage.

In some aspects, the system performs step simulation in a multi-step simulation, where the system can iteratively update and refine the positions, forces, and energies of atoms, ensuring the MD simulation remain accurate and physically realistic over the simulation time. By analyzing each step, the system can detect instability (if any), such as non-physical states or unrealistic behaviors, and fine-tune the MLFF model based on the detection.

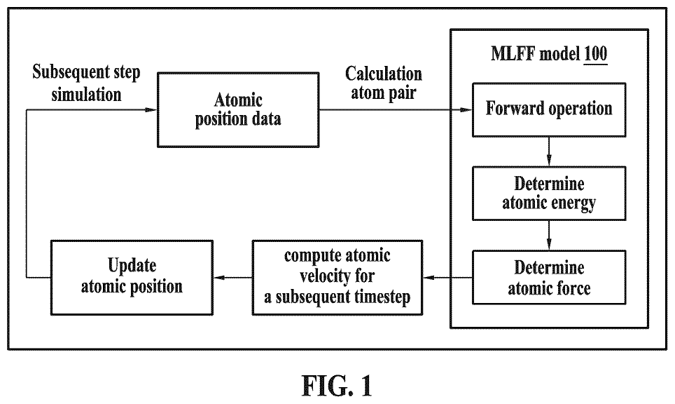

FIG. 1 is a diagram illustrating a process of performing MD simulation using an MLFF model 100 according to an embodiment of the present disclosure.

Referring to FIG. 1, in a simulation, an MLFF model 100 may receive input data (e.g., atomic position data) corresponding to a plurality of atoms. The MLFF model 100 may perform a forward operation on the atomic position data. The MLFF model 100 may generate atomic force (or force received by atoms) and atomic energy through the forward operation on the atomic position data. Then, the system computes an atomic velocity for a subsequent timestep based on the generated atomic force and the atomic energy. Then, the system updates the atomic position based on the computed atomic velocity. After the atomic position data is updated, the simulation may proceed to the subsequent step. For example, the system may generate atomic position data from the updated atomic position, and generate calculation atom pair. In some cases, the MLFF model 100 receives the calculated atom pairs and may perform a second forward operation to generate atomic energy and atomic force of the plurality of atom for the sequent timestep.

Evaluation indices for the MLFF model 100 may include metrics such as the force MAE of the atoms and the energy MAE of the atoms. However, in practical applications of MD simulation, the MLFF model 100 must be robust in various situations including learned data and unseen data distribution. Therefore, in addition to the accuracy (or precision) of the MLFF model, a framework for evaluating and enhancing the simulation stability is needed.

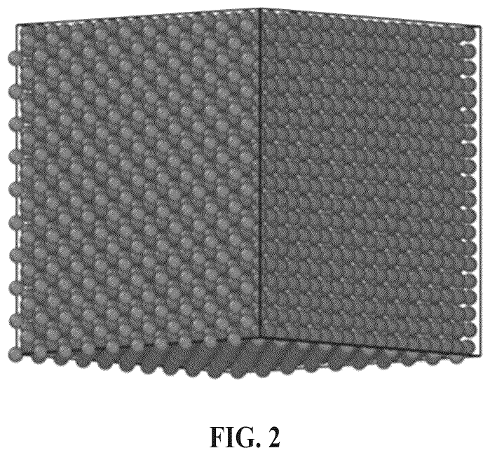

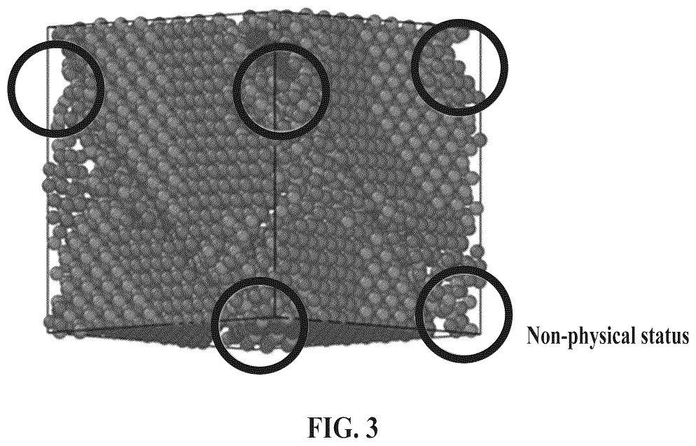

FIG. 2 is a diagram illustrating an atomic structure of hafnium oxide (HfO) before simulation according to an embodiment of the present disclosure. Referring to FIG. 2, a cube may be a simulation container (or a simulation box). The simulation container may include two types of atoms (e.g., hafnium atoms and oxygen atoms). FIG. 3 is a diagram illustrating an atomic structure of hafnium oxide (HfO) after simulation according to an example. For example, the simulation performed on the hafnium oxide (HfO) 40,000 steps. For example, an Allegro model may be used in the simulation.

Referring to FIG. 3, after simulating a predetermined timestep, gaps or blanks, such as the portions indicated by the circles, may be generated. Such blanks may be generated because the atoms originally located in the region indicated by the circles in the simulation container have flown out of the simulation container or the atoms have locally concentrated in other regions in the simulation container. As a result, the simulation may reach a non-physical status. Non-physical status may refer to as a situation where the simulation generates results that do not align with the fundamental law of physics.

In some cases, for example, Allegro-Legato or a GNN-based MLFF model with a simpler system architecture may achieve a relatively high simulation stability. However, the training time of these models may increase, or the prediction precision may decrease. Embodiments of the present disclosure provide a training method and training apparatus for an MLFF model that increase the simulation stability while reducing the training time and/or increase the prediction precision. The training method and training apparatus for an MLFF model according to embodiments of the present disclosure include training an MLFF model based on a correlation loss corresponding to edge features, thereby minimizing the correlation of edge features and improving simulation stability.

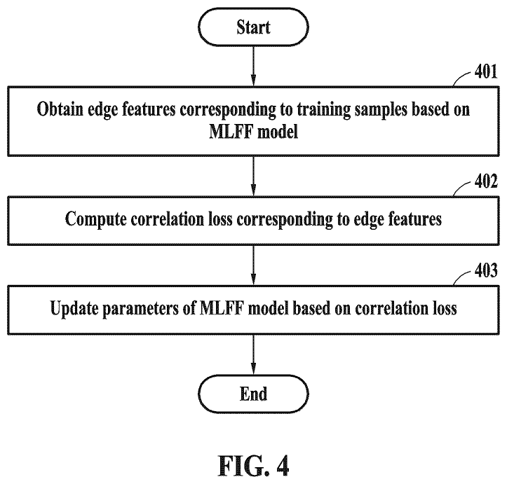

FIG. 4 is a flowchart illustrating a method for training an MLFF model according to an embodiment of the present disclosure. According to an embodiment, operations 401, 402, and 403 may be performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps or are performed in conjunction with other operations. The processor may include at least one processor including processing circuitry.

At operation 401, the system obtains edge features corresponding to training samples based on an MLFF model. For example, the training samples may include data related to a plurality of atoms. For example, the data related to the plurality of atoms may include the positions of atoms, the quantity of atoms, or the types of atoms, but is not limited thereto. The edge features may be features related to edges between the plurality of atoms. For example, the edge features may be in the form of vectors.

At operation 402, the system computes a correlation loss corresponding to the edge features. According to an embodiment, the MLFF model may include a plurality of neural network layers configured to obtain the training samples. In some embodiments, each layer of the plurality of neural network layers generates an edge feature matrix representing the edge features based on the training samples.

According to an embodiment, the system computes a correlation value between any two column vectors in the edge feature matrix for each layer. The system further computes a correlation loss corresponding to the edge feature matrix of each layer based on the correlation value.

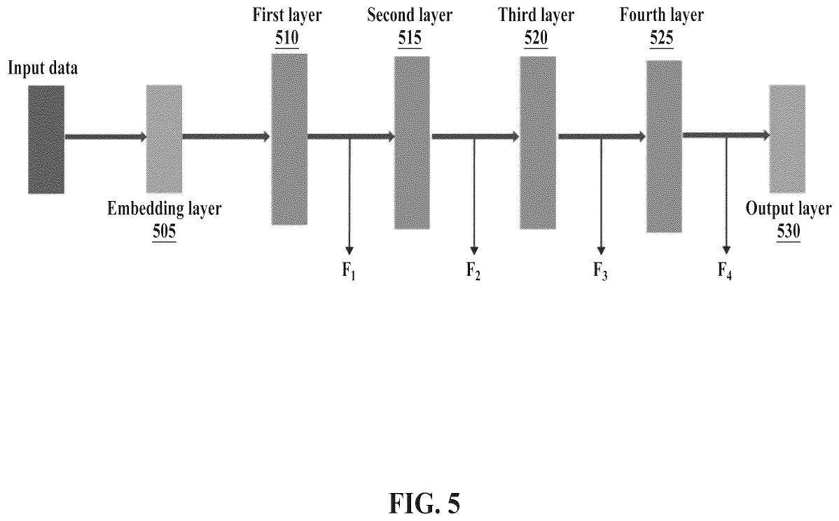

At operation 403, the system updates parameters of the MLFF model based on the correlation loss. Further detail on operation 403 is described with reference to FIG. 5.

FIG. 5 is a diagram illustrating a plurality of layers of an MLFF model according to an embodiment of the present disclosure. Referring to FIG. 5, an MLFF model may include an embedding layer 505, an output layer 530, and four GNN layers, which includes a first layer 510, a second layer 515, a third layer 520, and a fourth layer 525.

According to an embodiment, the embedding layer 505 receives the input data and generates an embedding based on the input data. For example, the input data includes data related to a plurality of atoms such as the positions of atoms, the quantity of atoms, or the types of atoms. For example, the embedding may represent the information from the input data in a numerical or vector/matrix representation for the MLFF model to process. The first layer 510 may generate an edge feature matrix F₁ based on the embedding, the second layer 515 may generate an edge feature matrix F₂ based on the edge feature matrix F₁, the third layer 520 may generate an edge feature matrix F₃ based on the edge feature matrix F₂, and the fourth layer 525 may generate an edge feature matrix F₄ based on the edge feature matrix F₃.

A graph neural network (GNN) is a type of neural network designed to operate on graph-structured data. In some cases, the GNN can process various sizes of graphs having different levels of complexities. For example, the nodes of the GNN represent entities depicted in the graph-structured data, and the edges of the GNN represent relationships between the entities depicted in the graph-structured data. In some aspects, a GNN uses the graph structure to aggregate and propagate information across nodes, and captures local and global patterns within the graph-structured data.

Among the plurality of GNN layers, an edge feature matrix Fᵢ output by an i-th layer may be represented in the form of [f, dim], where f represents the number of features, and dim represents the dimension of the features.

The system may compute the correlation value between any two column vectors included in the edge feature matrix from each layer using [Equation 1].

$$ρ ( {X}_{i} , {Y}_{i} ) = \frac{{\sum }_{i = 1}^{f} ( {X}_{i} - \overline{X} ) ( {Y}_{i} - \overline{Y} )}{\sqrt{{\sum }_{i = 1}^{f} {( {X}_{i} - \overline{X} )}^{2}} \sqrt{{\sum }_{i = 1}^{f} {( {Y}_{i} - \overline{Y} )}^{2}}} \qquad [ Equation 1 ]$$

where Xᵢ and Yᵢ are two different column vectors in the edge feature matrix Fᵢ, X̅ represents the average value of multiple elements in the column vector Xᵢ, and Y̅ represents the average value of multiple elements in the column vector Yᵢ.

According to an embodiment, the system may determine a correlation matrix Corr; corresponding to the edge feature matrix for each layer based on the calculated correlation value. The system may compute a correlation loss loss_{corr}ⁱ corresponding to the edge feature matrix for each layer based on the correlation matrix Corrᵢ corresponding to the edge feature matrix for each layer and a predetermined diagonal matrix.

The system may determine the correlation matrix Corrᵢ of the edge feature matrix Fᵢ of the i-th layer using [Equation 2].

$${Corr}_{i} [ k , j ] = \vert ρ ( {F}_{i} ( : , k ) , {F}_{i} ( : , j ) ) \vert \qquad [ Equation 2 ]$$

The correlation matrix Corrᵢ may be represented as [dim, dim]. Corrᵢ[k,j] represents an element in the k-th row and j-th column of the correlation matrix Corrᵢ, and ρ(Fᵢ(:,k), Fᵢ(:,j)) may represent a correlation value between the k-th column vector and the j-th column vector of the edge feature matrix Fᵢ of the i-th layer in the correlation matrix Corrᵢ.

For example, a correlation matrix with dim=3 may be represented as:

$${Corr}_{example} = [ \begin{matrix}1 & 0.2 & 0.1 \\ 0.2 & 1 & 0.5 \\ 0.1 & 0.5 & 1\end{matrix} ]$$

The correlation matrix Corrₑₓₐₘₚₗₑ may be a 3×3 matrix. The element “0.2” in the 0-th row and 1st column of the correlation matrix Corrₑₓₐₘₚₗₑ may be obtained by computing the correlation value ρ(Fᵢ(:,0), Fᵢ(:,1)) between the 0-th column vector and the 1st column vector of the edge feature matrix Fᵢ of the i-th layer. The element “0.1” in the 0-th row and 2nd column of the correlation matrix Corrₑₓₐₘₚₗₑ may be obtained by computing the correlation value ρ(Fᵢ(:,0), Fᵢ(:,2)) between the 0-th column vector and the 2nd column vector of the edge feature matrix Fᵢ of the i-th layer. The element “0.5” in the 1st row and 2nd column of the correlation matrix Corrₑₓₐₘₚₗₑ may be obtained by computing the correlation value ρ(Fᵢ(:,1), Fᵢ(:,2)) between the 1st column vector and the 2nd column vector of the edge feature matrix Fᵢ of the i-th layer.

According to some embodiments, the elements of the correlation matrix Corrₑₓₐₘₚₗₑ may be computed using a different index. For example, the element “0.2” in the first row and second column of the correlation matrix Corrₑₓₐₘₚₗₑ may be obtained by computing the correlation value ρ(Fᵢ(:,1), F; (:,2)) between the first column vector and the second column vector of the edge feature matrix Fᵢ of the i-th layer. The element “0.1” in the first row and third column of the correlation matrix Corrₑₓₐₘₚₗₑ may be obtained by computing the correlation value ρ(Fᵢ(:,1), Fᵢ(:,3)) between the first column vector and the third column vector of the edge feature matrix Fᵢ of the i-th layer.

According to an embodiment, to minimize the feature correlation for each layer, the optimization objective of the correlation matrix Corrᵢ may be a diagonal matrix with diagonal elements of “1”:

$${Corr}_{target} = [ \begin{matrix}1 & 0 & 0 \\ 0 & 1 & 0 \\ 0 & 0 & 1\end{matrix} ]$$

In some cases, the system may compute the correlation loss

$${loss}_{corr}^{i}$$

corresponding to the edge feature matrix of each layer using [Equation 3].

$${loss}_{corr}^{i} = \frac{\sum \vert {Corr}_{i} - {Corr}_{target} \vert}{f ( f - 1 )} \qquad [ Equation 3 ]$$

where Corr_{target} represents a predetermined diagonal matrix.

As described above, the system may compute a correlation loss corresponding to the edge feature matrix of each of the plurality of layers in the MLFF model. The sum of correlation losses of all layers in the MLFF model may be represented as shown in [Equation 4].

$${loss}_{corr} = {\sum }_{i} {loss}_{corr}^{i} \qquad [ Equation 4 ]$$

At operation 403, the system updates parameters of the MLFF model based on the correlation loss. For example, the trained MLFF model enhances the simulation stability and accuracy of molecular dynamics (MD) simulation using the trained MLFF model. This is achieved using the correlation loss to update parameters of the MLFF model. For example, the correlation loss minimizes the correlation between edge features in the MLFF model. Edge features represent relationships between atoms in the simulated system. When these features are highly correlated, the model may “overfit” or become less robust, which can lead to unstable predictions during simulations. Additionally, to perform MD simulation, precise output prediction is needed so that the system avoids generating a simulation with non-physical status (as shown in FIG. 3). By minimizing the correlation loss, the system improves the simulation stability.

According to an embodiment, the system updates the parameters of the MLFF model based on the correlation loss corresponding to the edge feature matrix of each of the plurality of layers corresponding to the training samples. The system may train the MLFF model by updating the parameters of the MLFF model based on the sum of correlation losses corresponding to respective edge feature matrices of all the plurality of layers included in the MLFF model.

There may be a negative correlation between the correlation of the edge features and the simulation stability of the GNN-based MLFF model. To enhance the simulation stability of a deep MLFF model, a correlation loss function may be used to reduce the correlation of the edge features in the MLFF model training. The optimization goal of the present disclosure is to minimize the correlation of the edge features. For example, any two column vectors within the edge feature matrix may be completely uncorrelated, and thus, the correlation value between any two column vectors is “0”. Therefore, when training the MLFF by setting the optimization goal of the correlation matrix to a diagonal matrix, the correlation of the edge features can be minimized, thereby enhancing the simulation stability of the MLFF model.

According to an embodiment, the system may compute the total training loss of the MLFF model based on the correlation loss, a loss corresponding to the force of the plurality of atoms (or the force received by the plurality of atoms), and a loss corresponding to the energy of the plurality of atoms. The system may further train the MLFF model by updating the parameters of the MLFF model based on the total training loss.

According to an embodiment, the data related to the plurality of atoms may include the real force Fᵣₑₐₗ of the plurality of atoms (or the real force received by the plurality of atoms) and the real energy Eᵣₑₐₗ of the plurality of atoms. The system may extract predicted force F_{predicted} and predicted energy E_{predicted} of the plurality of atoms from the training samples using the MLFF model.

The system may calculate a loss loss_{force} corresponding to the force of the plurality of atoms based on predicted force F_{predicted} and real force Fᵣₑₐₗ of each of the plurality of atoms. For example, the system may compute, as the loss loss_{force} corresponding to the force of the plurality of atoms, a mean absolute error (force MAE) of the atoms based on the predicted force F_{predicted} and the real force Fᵣₑₐₗ of each of the plurality of atoms.

The system may compute a loss loss_{energy} corresponding to the energy of the plurality of atoms based on predicted energy E_{predicted} and the real energy Eᵣₑₐₗ of each of the plurality of atoms. For example, the system may compute, as the loss loss_{energy} corresponding to the energy of the plurality of atoms, an energy MAE of the atoms based on the predicted energy E_{predicted} and the real energy Eᵣₑₐₗ of each of the plurality of atoms.

According to an embodiment, the system may determine a weight

$${coef}_{corr}^{{e}_{i}}$$

corresponding to the correlation loss, to calculate the total training loss of the MLFF model, for the MLFF model pretrained according to at least one epoch (or training epoch).

The MLFF model may be trained during a plurality of epochs. In different epochs of training, the same training samples may be used. For example, a total of “200” epochs of training may be performed, and each epoch of training may include “100” training samples. In such a manner, the MLFF model may be trained a total of “20,000” times. Each epoch of training may have a weight corresponding to each correlation loss (or a correlation loss weight

$${coef}_{corr}^{{e}_{i}} ) ,$$

and thus, correlation loss weights

$${coef}_{corr}^{{e}_{i}}$$

corresponding to different epochs of training may be different from each other. The plurality of training samples within the same training epoch may use the correlation loss weight

$${coef}_{corr}^{{e}_{i}}$$

corresponding to the training epoch.

The system may compute a weighted correlation loss based on the weight

$${coef}_{corr}^{{e}_{i}}$$

corresponding to the correlation loss and the correlation loss loss_{corr}. The system may compute the total training loss based on the weighted correlation loss, the loss corresponding to the force of the plurality of atoms, and the loss corresponding to the energy of the plurality of atoms.

According to an embodiment, to compensate for a decrease in the accuracy of the MLFF model due to the use of a correlation loss, the system may compute the correlation loss weight corresponding to each of the different training epochs. The correlation loss weight may also be referred to as the correlation loss coefficient

$${coef}_{corr}^{{e}_{i}} .$$

The system may use the correlation loss coefficient from each training epoch to optimize the loss of the training epoch. Accordingly, by using the correlation loss coefficient, the system may reduce the effect of the correlation loss on the training of the MLFF model, and may reduce the degree of decrease in the accuracy of the MLFF model in predicting the force of the atoms and the energy of the atoms. Accordingly, the training method of the present disclosure may improve the simulation stability of the MLFF model while preventing a decrease in the accuracy of the MLFF model.

In some aspects, the correlation loss coefficient improves the simulation stability of the MLFF model by dynamically updating the weights of the correlation loss during training. For example, during the early training stage, higher weights are used to minimize feature correlation to increase simulation stability, while lower weights in later epochs (or later training stage) focus on increasing the accuracy in force and energy predictions. Accordingly, the training method of the present disclosure improves the simulation stability of the MLFF model while preventing a decrease in the accuracy of the MLFF model by using the dynamic weights during the training stage.

According to an embodiment, the system may determine the weight corresponding to the correlation loss (or the correlation loss weight)

$${coef}_{corr}^{{e}_{i}}$$

for each training epoch based on the number of trained epochs, a predetermined initial weight coefᵢₙᵢₜ for the correlation loss, and predetermined update interval(s) for the correlation loss. For example, in a case where a total of “200” epochs of training is performed, the value of the training epoch may be an integer between “1” to “200”. If the number of trained epochs is “0”, for example, if training has not yet started, the value of the current training epoch may be expressed by eᵢ=1. If the number of trained epochs is “1”, for example, the first epoch of training has been completed, the value of the current training epoch may be expressed by eᵢ=2. If the number of trained epochs is “5”, for example, the fifth epoch of training has been completed, the value of the current training epoch may be expressed by eᵢ=6.

According to an embodiment, before each training epoch starts, the system may obtain the correlation loss weight of the current training epoch, for example, the correlation loss coefficient

$${coef}_{corr}^{{e}_{i}}$$

using [Equation 5].

$${coef}_{corr}^{{e}_{i}} = {coef}_{init} * \frac{{e}_{i}}{s} \qquad [ Equation 5 ]$$

where eᵢ represents the value of the current training epoch, and s represents the number of training samples.

After completing one forward operation using the MLFF model, the system may compute the total training loss using [Equation 6].

$$loss = {coef}_{force} * {loss}_{force} + {coef}_{energy} * {loss}_{energy} + {coef}_{corr}^{{e}_{i}} * {loss}_{corr} \qquad [ Equation 6 ]$$

where coef_{force} is a predetermined constant that represents a force loss coefficient, loss_{force} represents the loss corresponding to the force of the plurality of atoms, coef_{energy} is a predetermined constant that represents an energy loss coefficient, loss_{energy} represents the loss corresponding to the energy of the plurality of atoms,

$${coef}_{corr}^{{e}_{i}}$$

represents the correlation loss weight (or the correlation loss coefficient) of the current training epoch, and loss_{corr} represents the correlation loss.

The system may update the weight (or adjust the parameters) of the MLFF model based on the calculated total training loss. The system may perform a feedback operation on the MLFF model to train the MLFF model.

According to an embodiment, the system may generate a simulation output of a simulation tool for simulating the MLFF model, after updating the parameters of the MLFF model. For example, the simulation tool may be a large-scale atomic/molecular massively parallel simulator (LAMMPS), but is not limited thereto.

The system may compute the value of a simulation stability index S_{index} of the MLFF model based on the simulation output. According to an embodiment, the system may generate the value of the simulation stability index of the MLFF model. According to an embodiment, the system may generate evaluation information about the simulation stability of the MLFF model based on the value of the simulation stability index. The system may generate the evaluation information about the simulation stability of the MLFF model based on the value of the simulation stability index S_{index}. According to an embodiment, the system may also output the value of the simulation stability index and the evaluation information about simulation stability at the same time. The greater the value of the simulation stability index S_{index}, the higher the simulation stability of the MLFF model.

In some cases, the simulation stability index provides a quantitative measure of the physical validity of the MD simulation performed using the trained MLFF model. By incorporating the stability index into the training process, the system ensures that the MLFF model achieves high predictive accuracy and maintain reliability and robustness during the simulations.

As described above, the method for training a MLFF model according to an embodiment may accurately evaluate the simulation stability of the MLFF model. This is achieved by using the force MAE of the atoms and the energy MAE of the atoms as the main indicators for evaluating the MLFF model. In some cases, these indicators are used to evaluate the stability of MD simulation, and provide a more accurate and realistic reflection of the simulation stability of the MLFF model.

According to an embodiment, the system may generate a simulation output for each simulation step, in the multi-step simulation performed continuously by the simulation tool. For example, the system may execute q-step MD simulation using the MLFF model trained based on at least one epoch, through the simulation tool such as an LAMMPS. The simulation output for each simulation step may include the simulation positions of atoms posₐₜₒₘ and the simulation quantity of the atoms atom. The system may compute the value of the simulation stability index S_{index} based on the simulation output of each step simulation, in the multi-step simulation.

The system may store the simulation output, during the simulation process, at intervals of Δt. For example, if Δt=100 in a total of “4,000” simulations, the processor may store (or record) the simulation output of the current simulation every “100” simulations. In some embodiments, every simulation output may be stored. The system may record trajectory data of the current simulation, for example, simulation outputs of a total of “40” simulations, which are the simulation output of the 100-th simulation, the simulation output of the 200-th simulation, . . . , the simulation output of the 500-th simulation, . . . , the simulation output of the 3900-th simulation, and the simulation output of the 4000-th simulation. The system may determine the simulation stability of the MLFF model by computing the value of the simulation stability index S_{index} using the recorded “40” simulation outputs.

The system may generate the simulation output for each step simulation as described. For example, the system may generate an atomic force state (or a state of force received by the atom) by inputting the simulation output of a previous step simulation into the trained MLFF model. For example, the system may generate the atomic force state by inputting the simulation positions of the atoms posₐₜₒₘ and the simulation quantity of the atoms atom, included in the simulation output of the previous step simulation, into the trained MLFF model. The previous step simulation may be a step simulation immediately before each step simulation.

The system may obtain the simulation output of each step simulation by inputting the atomic force state into the simulation tool. The simulation tool may determine the position posₐₜₒₘ and the number atom of the plurality of atoms in the subsequent timestep based on the atomic force state.

According to an embodiment, the simulation output may include the simulation positions of the atoms posₐₜₒₘ, the simulation quantity of the atoms atom, and the temperature in a simulation container. The simulation container may be a container for accommodating atoms, and the temperature in the simulation container may be determined based on the temperatures of the plurality of atoms in the container.

As described above, the system may obtain the simulation output for each step simulation in the multi-step simulation performed continuously using the simulation tool. The simulation output of each step simulation may include the simulation positions of the atoms posₐₜₒₘ, the simulation quantity of the atoms atom, and the temperature in the simulation container TnΔt. Referring to FIG. 2 or 3, the shown cube may be the simulation container or the simulation box.

In some aspects, by performing each step simulation in a multi-step simulation, the system can iteratively update and refine the positions, forces, and energies of atoms, ensuring the MD simulation remain accurate and physically realistic over the simulation time. By analyzing each step, the system can detect instability (if any), such as non-physical states or unrealistic behaviors, and fine-tune the MLFF model based on the detection. Additionally, by performing step-by-step simulations, the system can generate intermediate outputs (e.g., atomic positions, radial distribution functions, and temperatures), which can be used to further compute the simulation stability index.

The system may compute a radial distribution function (RDF) value for each of a plurality of atom pairs in a simulation based on the simulation positions of the atoms posₐₜₒₘ and the simulation quantity of the atoms atom in the simulation output. For example, the system may compute the RDF for each of the plurality of atom pairs in each step simulation based on the simulation positions of the atoms posₐₜₒₘ and the simulation quantity of the atoms atom in the simulation output of each step simulation among the multi-step simulation performed continuously using the simulation tool. Each of the plurality of atom pairs may be an atom pair including any two atoms.

For example, the system may compute the RDF corresponding to each simulation based on the simulation positions of the atoms posₐₜₒₘ and the simulation quantity of the atoms atom included in each of the previously stored “40” simulation outputs. For example, each of the “40” simulations may correspond to each RDF.

For example, the system may compute the RDF values of different atom pairs using the RDF function shown in [Equation 7].

$$R D F ( r ) = \frac{1}{4 π {r}^{2}} \frac{1}{Nd} {\sum }_{i}^{N} {\sum }_{j \neq i}^{N} δ ( r - ‖ {x}_{i} - {x}_{j} ‖ ) \qquad [ Equation 7 ]$$

where r represents the distance from a reference particles, and N represents the total number of particles, (e.g., the simulation quantity of the atoms in the simulation output of each step simulation). Additionally, I and j represent an atom pair, d represents the system density (e.g., the density of atoms in the simulation container). For example, d=N/V, where V represents the volume of the simulation container, and δ may be a Dirac delta function used to extract a value distribution.

The system may compute the value of the simulation stability index S_{index} based on the RDF value corresponding to the simulation, the simulation quantity of the atoms and the temperature in the simulation container TnΔt in the simulation output, and the initial quantity of atoms atomᵢₙᵢₜ before the simulation starts. For example, the system may compute the value of the simulation stability index S_{index} based on the RDF value corresponding to each step simulation, the simulation quantity of the atoms in the simulation output of each step simulation, the temperature in the simulation container TnΔt, and the initial quantity of atoms atomᵢₙᵢₜ before the simulation starts.

Accordingly, the system may compute the value of the simulation stability index S_{index} using [Equation 8].

$${s}_{index} = \frac{1}{num} {\sum }_{n = 1}^{num} ( {( \frac{{atom}_{n Δ t}}{{atom}_{init}} )}^{α} * {( \frac{{T}_{set}}{{T}_{n Δ t}} )}^{β} * {\prod }_{i = 0}^{m} ( {RDF}_{n Δ t}^{i} - {RDF}_{( n - 1 ) Δ t}^{i} ) ) \qquad [ Equation 8 ]$$

where num is the number of step simulations used for evaluation and represents the ratio between the total number of step simulations q and a simulation interval Δt. For example, num=q/Δt, and atom_{nΔt} represents the quantity of atoms in the simulation output of an n-th step simulation, atomᵢₙᵢₜ represents the initial quantity of atoms before the simulation starts, a may denote a scaling factor for the number of atoms. Tₛₑₜ represents a temperature set by a user in the simulation, TnΔt represents the temperature in the simulation container in the simulation output of the n-th step simulation, β represents a scaling factor for the temperature,

$$R D {F}_{n Δ t}^{i}$$

represents the RDF value of an i-th type of atom pair in the n-th step simulation,

$$R D {F}_{( n - 1 ) Δ t}^{i}$$

represents the RDF value of the i-th type of atom pair in an (n−1)-th step simulation, and m represents the total number of atom pair types.

For example, hafnium oxide (HfO), which is one of the materials used in semiconductor thin films, may include two types of atoms, namely oxygen atoms (O) and hafnium atoms (Hf). Hafnium oxide (HfO) may correspond to three types of atom pairs, namely an atom pair “Hf—O” including oxygen atoms (O) and hafnium atoms (Hf), an atom pair “O—O” including oxygen atoms (O) and oxygen atoms (O), and an atom pair “Hf—Hf” including hafnium atoms (Hf) and hafnium atoms (Hf).

The method for computing the simulation stability index S_{index} may not be necessarily limited to the method as in [Equation 8], and there may be other computational methods. For example, the system may determine the simulation stability index S_{index} based on any one of the RDF values corresponding to the simulation, the quantity of atoms atom included in the simulation output, and the temperature in the simulation container TnΔt. Alternatively, the system may determine the simulation stability index S_{index} based on any two of the RDF value, the quantity of atoms included in the simulation output, and the temperature in the simulation container TnΔt. For example, the system may use the product of any two of the foregoing as the simulation stability index S_{index}. The specific method of computing the simulation stability index is not limited to those of the present disclosure, and the methods are merely an exemplary description.

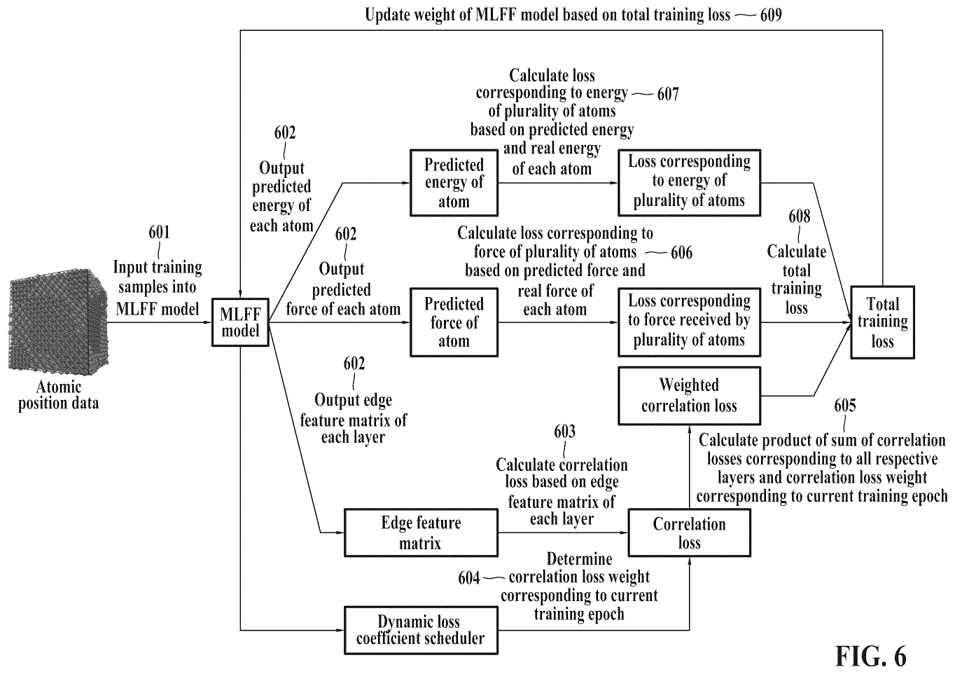

FIG. 6 is a diagram illustrating a method for training an MLFF model according to an embodiment of the present disclosure. Δt operation 601, the system may input training samples into an MLFF model. The training samples may include data related to a plurality of atoms, such as the positions of atoms Pos, the quantity of atoms, the types of atoms, the real force of the atoms Fᵣₑₐₗ, the real energy of the atoms Eᵣₑₐₗ, etc. Additionally, the system may input a predetermined initial weight coefᵢₙᵢₜ for a correlation loss and predetermined update interval(s) for a correlation loss into the MLFF model. In some cases, for example, the system is initialized based on the predetermined initial weight coefᵢₙᵢₜ and the predetermined update interval(s).

At operation 602, the MLFF model generates predicted force F_{predicted}=MLFF(Pos) and predicted energy E_{predicted}=MLFF(Pos) for each of the plurality of atoms in the training samples. In addition, the MLFF model generates an edge feature matrix Fᵢ=forward(Pos) for each of a plurality of layers (e.g., GNN layers) in the MLFF model. The system may generate the predicted force F_{predicted}=MLFF(Pos) and the predicted energy E_{predicted}=MLFF(Pos) of each of the plurality of atoms in the training samples, and the edge feature matrix Fᵢ=forward(Pos) of each of the plurality of layers (e.g., the GNN layers) included in the MLFF model.

At operation 603, the system may compute a correlation loss based on the edge feature matrix Fᵢ for each layer. The system may determine a correlation matrix Corrᵢ corresponding to the edge feature matrix Fᵢ for each layer of the MLFF model. The system may compute a correlation loss loss_{corr}ⁱ corresponding to the edge feature matrix of each layer, based on the correlation matrix Corrᵢ of each layer and a predetermined diagonal matrix (as described with reference to FIG. 5). The system may compute the sum of each correlation losses loss_{corr}ⁱ of each layer to obtain the total correlation loss (or correlation loss):

$${loss}_{corr} = {\sum }_{i} {loss}_{corr}^{i} .$$

At operation 604, the system may determine a correlation loss weight

$${coef}_{c o r r}^{{e}_{i}}$$

corresponding to the current training epoch. For example, the system may use a dynamic loss coefficient schedular to compute the correlation loss weight

$${coef}_{corr}^{{e}_{i}}$$

corresponding to the current training epoch based on the number of trained epochs, the predetermined initial weight coefᵢₙᵢₜ for a correlation loss, and the predetermined update interval(s) for a correlation loss.

For example, in a case where a total of “200” epochs of training is performed, the value of the training epoch may be an integer between “1” to “200”. If the number of trained epochs is “0”, then the value of the current training epoch may be expressed by eᵢ=1. In some cases, when the number of trained epochs is “0”, this indicates that the training has not yet started. If the number of trained epochs is “1”, (e.g., the first epoch of training has been completed), then the value of the current training epoch may be expressed by eᵢ=2. If the number of trained epochs is “5”, (e.g., the fifth epoch of training has been completed), then the value of the current training epoch may be expressed by eᵢ=6.

Before each training epoch starts, the system may obtain the correlation loss weight of the current training epoch, for example, the correlation loss coefficient

$${coef}_{corr}^{{e}_{i}}$$

using [Equation 5].

At operation 605, the system may compute a weighted correlation loss by computing the product of the sum loss_{corr} of correlation losses loss_{corr}ⁱ corresponding to all respective layers and the correlation loss weight

$${coef}_{corr}^{{e}_{i}}$$

corresponding to the current training epoch.

At operation 606, the system may compute a loss loss_{force}=MAE(Fᵣₑₐₗ, F_{predicted}) corresponding to the force of the plurality of atoms, based on predicted force F_{predicted} and real force Fᵣₑₐₗ of each of the plurality of atoms from the training samples. MAE represents the mean absolute error.

At operation 607, the system may compute a loss loss_{energy} MAE(Eᵣₑₐₗ, E_{predicted}) corresponding to the energy of the plurality of atoms based on predicted energy E_{predicted} and real energy Eᵣₑₐₗ of each of the plurality of atoms from the training samples.

At operation 608, the system may compute a total training loss based on the weighted correlation loss, a loss corresponding to the force of the plurality of atoms, and a loss corresponding to the energy of the plurality of atoms. For example, the system may compute the total training loss

$$loss = {coef}_{force} * {loss}_{force} + {coef}_{energy} * {loss}_{energy} + {coef}_{corr}^{{e}_{i}} * {loss}_{corr} s$$

using [Equation 6]. For example, coef_{force} is a predetermined constant that represents a force loss coefficient, loss_{force} represents the loss corresponding to the force of the plurality of atoms, coef_{energy} is a predetermined constant that represents an energy loss coefficient, loss_{energy} represents the loss corresponding to the energy of the plurality of atoms,

$${coef}_{corr}^{{e}_{i}}$$

represents the correlation loss weight (or the correlation loss coefficient) of the current training epoch, and loss_{corr} represents the correlation loss.

At operation 609, the system may update the weight (or adjust the parameters) of the MLFF model based on the total training loss loss, and training the MLFF model using the total training loss.

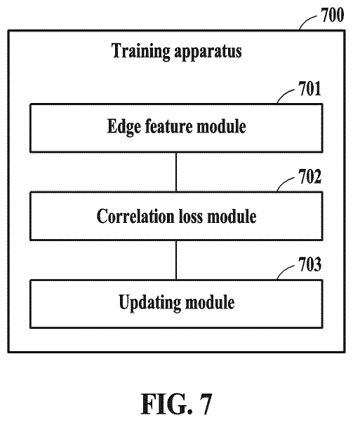

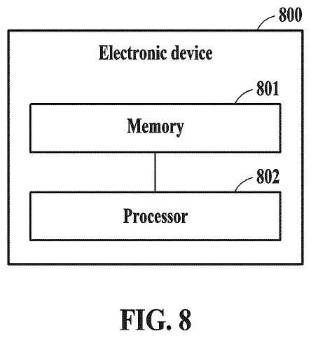

FIG. 7 is a block diagram of a training apparatus of an MLFF model according to an embodiment of the present disclosure. The training apparatus 700 may include an edge feature module 701, a correlation loss module 702, and an updating module 703. In some cases, each of the edge feature module 701, correlation loss module 702, and updating module 703 may be implemented as software stored in a memory unit (e.g., the memory 801 described with reference to FIG. 8) and executable by a processor unit (e.g., the processor 802 described with reference to FIG. 8), as firmware, as one or more hardware circuits, or as a combination thereof.

The edge feature module 701 may obtain edge features corresponding to training samples using an MLFF model. The training samples may include data related to a plurality of atoms. For example, the data related to the plurality of atoms may include the positions of atoms, the quantity of atoms, or the types of atoms, but is not limited thereto. The edge features may be features related to edges between the plurality of atoms. For example, the edge features may be in the form of vectors.

The correlation loss module 702 may compute a correlation loss corresponding to the edge features. According to an embodiment, the edge feature module 701 may generate an edge feature matrix for each of a plurality of layers (e.g., GNN layers) in the MLFF model, based on the edge features and the training samples. The correlation loss module 702 may compute a correlation value between any two column vectors in the edge feature matrix for each layer. The correlation loss module 702 may compute a correlation loss corresponding to the edge feature matrix of each layer based on the correlation value. Further detail on computing a correlation loss is described with reference to FIGS. 5 and 6.

According to an embodiment, the correlation loss module 702 may determine a correlation matrix Corrᵢ corresponding to the edge feature matrix of each layer based on the calculated correlation value. The correlation loss module 702 may compute a correlation loss loss_{corr} corresponding to the edge feature matrix of each layer based on the correlation matrix Corrᵢ corresponding to the edge feature matrix of each layer and a predetermined diagonal matrix (e.g., an identity matrix or a unit matrix where the elements of the diagonal are “1”s. Further detail on determining the correlation matrix is described with reference to FIGS. 5 and 6.

The updating module 703 may update parameters of the MLFF model based on the correlation loss. For example, the updating module 703 may update the parameters of the MLFF model based on the correlation loss corresponding to the edge feature matrix of each of the plurality of layers corresponding to the training samples. The updating module 703 may train the MLFF model by updating the parameters of the MLFF model based on the sum of correlation losses corresponding to respective edge feature matrices of all the plurality of layers in the MLFF model. Further detail on updating parameters of the MLFF model is described with reference to FIGS. 5 and 6.

In some cases, there may be a negative correlation between the correlation of the edge features and the simulation stability of the GNN-based MLFF model. To enhance the simulation stability of a deep MLFF model, a correlation loss function may be provided to reduce the correlation of the edge features in the MLFF model training. The optimization goal of the present disclosure is to minimize the correlation of the edge features. For example, in the ideal case where any two column vectors in the edge feature matrix are completely uncorrelated, the correlation value between any two column vectors is “0”. Therefore, by setting the optimization goal of the correlation matrix to a diagonal matrix, the correlation of the edge features can be minimized when training the MLFF model, thereby increasing the simulation stability of the MLFF model.

According to an embodiment, the updating module 703 may compute the total training loss of the MLFF model based on the correlation loss, a loss corresponding to the force of the plurality of atoms (or the force received by the plurality of atoms), and a loss corresponding to the energy of the plurality of atoms. The updating module 703 may train the MLFF model by updating the parameters of the MLFF model based on the total training loss. Further detail on computing the total training loss is described with reference to FIGS. 5 and 6.

According to an embodiment, the training apparatus 700 may further include a prediction module, a force loss calculating module, and an energy loss calculating module. In some aspects, each of the prediction module, force loss calculating module, and energy loss calculating module may be implemented as software stored in a memory unit (e.g., the memory 801 described with reference to FIG. 8) and executable by a processor unit (e.g., the processor 802 described with reference to FIG. 8), as firmware, as one or more hardware circuits, or as a combination thereof.

The data related to the plurality of atoms may include the real force Fᵣₑₐₗ of the plurality of atoms (or the real force received by the plurality of atoms) and the real energy Eᵣₑₐₗ of the plurality of atoms. The prediction module may generate predicted force F_{predicted} and predicted energy E_{predicted} of the plurality of atoms based on the training samples using the MLFF model.

The force loss calculating module may compute a loss loss_{force} corresponding to the force of the plurality of atoms based on predicted force F_{predicted} and real force Fᵣₑₐₗ of each of the plurality of atoms. For example, the force loss calculating module may compute, as the loss loss_{force} corresponding to the force of the plurality of atoms, a mean absolute error (force MAE) of the atoms based on the predicted force F_{predicted} and the real force Fᵣₑₐₗ of each of the plurality of atoms. Further detail on the method for computing the loss corresponding to the force of the plurality of atoms is described with reference to FIG. 5

The energy loss calculating module may compute a loss loss_{energy} corresponding to the energy of the plurality of atoms based on predicted energy E_{predicted} and the real energy Eᵣₑₐₗ of each of the plurality of atoms. For example, the energy loss calculating module may compute, as the loss loss_{energy} corresponding to the energy of the plurality of atoms, an energy MAE of the atoms based on the predicted energy E_{predicted} and the real energy Eᵣₑₐₗ of each of the plurality of atoms. Further detail on the method for computing the loss corresponding to the energy of the plurality of atoms is described with reference to FIG. 5.

According to an embodiment, the training apparatus 700 may further include a weight determining module. In one aspect, the weight determining module is implemented as software stored in a memory unit (e.g., the memory 801 described with reference to FIG. 8) and executable by a processor unit (e.g., the processor 802 described with reference to FIG. 8), as firmware, as one or more hardware circuits, or as a combination thereof. The weight determining module may determine a weight

$${coef}_{corr}^{{e}_{i}}$$

corresponding to the correlation loss, to compute the total training loss of the MLFF model, for the MLFF model pretrained according to at least one epoch (or training epoch).

The MLFF model may be trained during a plurality of epochs. In different epochs of training, the same training samples may be used. For example, a total of “200” epochs of training may be performed, and each epoch of training may include “100” training samples. As a result, the MLFF model may be trained a total of “20,000” times. Each epoch of training may have a weight corresponding to each correlation loss (or a correlation loss weight

$${coef}_{corr}^{{e}_{i}} ) ,$$

and thus, correlation loss weights

$${coef}_{corr}^{{e}_{i}}$$

corresponding to different epochs of training may be different from each other. The plurality of training samples within the same training epoch may use the correlation loss weight

$${coef}_{corr}^{{e}_{i}}$$

corresponding to the training epoch.

The updating module 703 may compute a weighted correlation loss based on the weight

$${coef}_{corr}^{{e}_{i}}$$

corresponding to the determined correlation loss and the correlation loss loss_{corr}. The updating module 703 may compute the total training loss based on the weighted correlation loss, the loss corresponding to the force of the plurality of atoms, and the loss corresponding to the energy of the plurality of atoms.

To compensate for a decrease in the accuracy of the MLFF model due to the use of a correlation loss, the training apparatus 700 may compute the correlation loss weight corresponding to each of the different training epochs. The correlation loss weight may also be referred to as the correlation loss coefficient

$${coef}_{corr}^{{e}_{i}} .$$

The system may use the correlation loss coefficient of each training epoch to optimize the loss of the training epoch. This may reduce the effect of the correlation loss on the training of the MLFF model, and may reduce the degree of decrease in the accuracy of the MLFF model in predicting the force of the atoms and the energy of the atoms. Accordingly, the training method of the present disclosure may increase the simulation stability of the MLFF model while preventing a decrease in the accuracy of the MLFF model.

According to an embodiment, the weight determining module may determine the weight corresponding to the correlation loss (or the correlation loss weight)

$${coef}_{corr}^{{e}_{i}}$$

of each training epoch based on the number of trained epochs, a predetermined initial weight coefᵢₙᵢₜ for the correlation loss, and predetermined update interval(s) for the correlation loss. For example, in a case where a total of “200” epochs of training is performed, the value of the training epoch may be an integer between “1” to “200”. If the number of trained epochs is “0”, for example, when training has not yet started, then the value of the current training epoch may be expressed by eᵢ=1. If the number of trained epochs is “1”, (e.g., the first epoch of training has been completed), then the value of the current training epoch may be expressed by eᵢ=2. If the number of trained epochs is “5”, (e.g., the fifth epoch of training has been completed), then the value of the current training epoch may be expressed by eᵢ=6.

According to an embodiment, the training apparatus 700 may further include a simulation output obtaining module, a simulation stability index calculating module, and an output module. Each of the simulation output obtaining module, simulation stability index calculating module, and output module may be implemented as software stored in a memory unit (e.g., the memory 801 described with reference to FIG. 8) and executable by a processor unit (e.g., the processor 802 described with reference to FIG. 8), as firmware, as one or more hardware circuits, or as a combination thereof.

After the parameters of the MLFF model are updated, the simulation output obtaining module may obtain a simulation output of a simulation tool for simulating the MLFF model. For example, the simulation tool may be an LAMMPS, but is not limited thereto.

The simulation stability index calculating module may compute the value of a simulation stability index S_{index} of the MLFF model based on the simulation output. According to an embodiment, the output module may output the value of the simulation stability index of the MLFF model based on the calculated value of the simulation stability index. According to an embodiment, the output module may output evaluation information about the simulation stability of the MLFF model based on the value of the simulation stability index. According to an embodiment, the output module may also output the value of the simulation stability index and the evaluation information about simulation stability at the same time. The greater the value of the simulation stability index S_{index}, the higher the simulation stability of the MLFF model.

As described above, the training apparatus 700 may accurately evaluate the simulation stability of the MLFF model by using the force MAE of the atoms and the energy MAE of the atoms as the main indicators for evaluating the MLFF model. In some cases, the training apparatus 700 is used to evaluate the stability of MD simulation, and may reflect the simulation stability of the MLFF model more accurately and realistically.

According to an embodiment, the simulation output obtaining module may obtain a simulation output of each step simulation among the multi-step simulation performed continuously using the simulation tool. For example, the simulation output obtaining module may execute q-step MD simulation using the MLFF model trained according to at least one epoch, through the simulation tool such as an LAMMPS. The simulation output of each step simulation may include the simulation positions of atoms posₐₜₒₘ and the simulation quantity of the atoms atom. The simulation stability index calculating module may compute the value of the simulation stability index S_{index} based on the simulation output of each step simulation, in the multi-step simulation.

The simulation output of each step simulation may be obtained as described below. The simulation output obtaining module may obtain an atomic force state (or a state of force received by the atom) by inputting the simulation output of a previous step simulation into the trained MLFF model. For example, the simulation output obtaining module may obtain the atomic force state by inputting the simulation positions of the atoms posₐₜₒₘ and the simulation quantity of the atoms atom, included in the simulation output of the previous step simulation, into the trained MLFF model. The previous step simulation may be a step simulation immediately before each step simulation. The simulation output obtaining module may obtain the simulation output of each step simulation by inputting the atomic force state into the simulation tool. The simulation tool may determine the positions posₐₜₒₘ and the quantity atom of the plurality of atoms in the subsequent timestep based on the atomic force state.

According to an embodiment, the simulation output may include the simulation positions of the atoms posₐₜₒₘ, the simulation quantity of the atoms atom, and the temperature in a simulation container. The simulation container may be a container for accommodating atoms, and the temperature in the simulation container may be determined based on the temperatures of the plurality of atoms in the container.

The simulation stability index calculating module may compute an RDF value of each of a plurality of atom pairs in a simulation based on the simulation positions of the atoms posₐₜₒₘ and the simulation quantity of the atoms in the simulation output. For example, the simulation stability index calculating module may compute the RDF of each of the plurality of atom pairs in each step simulation, based on the simulation positions of the atoms posₐₜₒₘ and the simulation quantity of the atoms atom in the simulation output of each step simulation among the multi-step simulation performed continuously using the simulation tool. Each of the plurality of atom pairs may be an atom pair including any two atoms. Further detail on computing the RDF value is described with reference to FIG. 5.

The simulation stability index calculating module may compute the value of the simulation stability index S_{index} based on the RDF value corresponding to the simulation, the simulation quantity of the atoms atom and the temperature in the simulation container TnΔt included in the simulation output, and the initial quantity of atoms atomᵢₙᵢₜ before the simulation starts. Further detail on computing the value of the simulation stability index is described with reference to FIG. 5.

FIG. 8 is a block diagram of an electronic device according to an embodiment of the present disclosure. An electronic device 800 according to an embodiment may include at least one memory 801 and at least one processor 802 including processing circuitry. The at least one memory 801 may include one or more storage media configured to store instructions. The instructions, when executed by the at least one processor 802 individually or collectively, may cause the electronic device 800 to perform the training method of the present disclosure.

The electronic device 800 may be a personal computer (PC), a tablet device, a personal digital assistant (PDA), a smartphone, or other device capable of executing the instructions. The electronic device 800 is not necessarily a single electronic device, but may also be any device or circuit collection capable of executing the instructions (or instruction sets) individually or collectively. The electronic device 800 may be part of an integrated control system or system manager, or may be configured as a portable electronic device that interfaces locally or remotely (e.g., through wireless transmission).

In the electronic device 800, the at least one processor 1020 may include a central processing unit (CPU), a graphics processing unit (GPU), a programmable logic device, a dedicated processor system, a microcontroller, or a microprocessor. As a non-limiting example, the at least one processor 802 may further include an analog processor, a digital processor, a microprocessor, a multi-core processor, a processor array, a network processor, or the like.

The at least one processor 802 may execute instructions or code stored in the at least one memory 801. The instructions and code may be transmitted and received over a network via a network interface that may employ any known transmission protocol, and a network interface device may utilize all known transmission protocols. For example, the instructions and code may be transmitted via a communication interface. The communication interface operates at a boundary between communicating entities (such as electronic device 800, one or more user devices, a cloud, and one or more databases) and can record and process communications. In some cases, the communication interface is provided to enable a processing system coupled to a transceiver (e.g., a transmitter and/or a receiver). In some examples, the transceiver is configured to transmit (or send) and receive signals for a communications device via an antenna. In some cases, a bus is used in communication interface.

The at least one memory 801 may be integrated with the at least one processor 802, for example, by arranging random-access memory (RAM) or flash memory within an integrated circuit microprocessor. In addition, the at least one memory 801 may include an independent device, such as an external disk drive, a storage array, or any other storage device that can be used in a database system. The at least one memory 801 and the at least one processor 802 may be operatively coupled, or may communicate with each other through an input/output (I/O) port, a network connection, or the like, so that the at least one processor 802 may read files stored in the at least one memory 801.

The electronic device 800 may further include, for example, a video display (e.g., a liquid crystal display) and a user interaction interface (e.g., a keyboard, a mouse, a touch input device, etc.). In some aspects, the components of the electronic device 800 may be connected to each other via a bus, a network, and/or a communication interface.

According to an embodiment, a non-transitory computer-readable storage medium may be provided. The non-transitory computer-readable storage medium may store one or more computer programs including instructions. When the instructions are executed individually or collectively by a CPU or a GPU, the instructions may cause the training method of the present disclosure to be performed. Examples of a non-transitory computer-readable storage medium may include read-only memory (ROM), random access programmable read-only memory (PROM), electrically erasable programmable read-only memory (EEPROM), random-access memory (RAM), dynamic RAM (DRAM), static RAM (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), card memory (e.g., a multimedia card, a secure digital (SD) card, or an extreme digital (XD) card), magnetic tapes, floppy disks, magneto-optical data storage devices, optical data storage devices, hard disks, solid-state disks, and any other device.

For example, other device may store computer programs and any associated data, data files, and data structures in a non-transitory manner and may provide the computer programs and any associated data, data files, and data structures to a processor or computer so that the processor or computer may execute the computer programs. The computer programs in the non-transitory computer-readable storage medium may run in an environment deployed in a computer device, such as a client, a host, a proxy device, a server, and the like. In an example, the computer programs and any associated data, data files, and data structures may be distributed over network-coupled computer systems so that the computer programs and any associated data, data files, and data structures may be stored, accessed, and executed in a distributed fashion by one or more processors or computers.

An electronic device according to an embodiment may further include a computer-readable storage medium storing a computer program. The computer program, when executed by a processor, may implement the training method as described in any one of the claims.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described hardware devices may be configured to act as one or more software modules to perform the operations of the above-described examples, or vice versa.

As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof.

Several embodiments of the present inventive concept have been described above. Nevertheless, it should be understood that various modifications may be made to these embodiments. For example, suitable results may be achieved if the described techniques are performed in a different order, and/or if components in a described system, architecture, device, or circuit are combined in a different manner, and/or replaced or supplemented by other components or their equivalents. Therefore, other implementations, other embodiments, and equivalents to the claims are also within the scope of the following claims.

One skilled in the art will readily conceive other embodiments of the present disclosure in consideration of the description and practice of the invention. The present disclosure is intended to cover any variations, uses, or adaptive changes that follow the general principles set forth herein and include common knowledge or customary techniques in the art that are not disclosed herein. The description and embodiments are considered as exemplary only, and the true scope and spirit of the present disclosure are defined by the claims set forth below.

It should be understood that the present disclosure is not limited to the exact structures described above and shown in the drawings, and that various modifications and changes can be made thereto without departing from the scope of the disclosure.

What is claimed is:

1. A method for training a machine learning force fields (MLFF) model, the method comprising:
obtaining, using the MLFF model, edge features corresponding to a training sample, wherein the training sample includes data related to a plurality of atoms, and the edge features represent relationship between edges of each atom among the plurality of atoms;
computing a correlation loss corresponding to the edge features, wherein the correlation loss represents correlation between edge features corresponding to the plurality of atoms;
updating parameters of the MLFF model based on the correlation loss to obtain a trained MLFF model; and
generating, using the trained MLFF model, a molecular dynamics (MD) simulation based on an input sample.

2. The method of claim 1, wherein updating parameters of the MLFF model comprises:
computing a total training loss based on the correlation loss, a force loss corresponding to force of the plurality of atoms, and an energy loss corresponding to energy of the plurality of atoms; and
updating the parameters of the MLFF model based on the total training loss.

3. The method of claim 2, wherein computing the total training loss comprises:
determining a first weight corresponding to the correlation loss in a first training timestep; and
computing a weighted correlation loss based on the first weight and the correlation loss, wherein the total training loss is computed based on the weighted correlation loss.

4. The method of claim 3, wherein determining the weight comprises:
determining a second weight corresponding to the correlation loss based on a second training timestep, a predetermined initial weight for a correlation loss in the second training timestep, and a predetermined update interval for a correlation loss in the second training timestep.

5. The method of claim 2, further comprising:
extracting real force and real energy of the plurality of atoms based on the training sample;
generating, using the trained MLFF model, predicted force and predicted energy for the plurality of atoms;
computing the force loss based on predicted force and real force; and
computing the energy loss based on predicted energy and real energy.

6. The method of claim 1, further comprising:
generating an edge feature matrix in each layer of a plurality of layers of the MLFF model based on the training sample;
computing a correlation value based on the edge feature matrix;
computing a correlation loss corresponding to the edge feature matrix at each layer based on the correlation value; and
updating parameters of the MLFF model based on the correlation loss.

7. The method of claim 6, wherein computing the correlation loss comprises:
generating a correlation matrix corresponding to the edge feature matrix at each layer based on the correlation value, wherein the correlation loss is computed based on the correlation matrix and a predetermined diagonal matrix.

8. The method of claim 1, further comprising:
obtaining a simulation output using the trained MLFF model;
computing a value of a simulation stability index of the MLFF model based on the simulation output; and
displaying the value of the simulation stability index.

9. The method of claim 8, further comprising:
obtaining simulation positions of atoms, a simulation quantity of atoms, and temperature in a simulation container based on the simulation output; computing a radial distribution function (RDF) value of each atom pair of the plurality of atom based on the simulation positions and the simulation quantity; and
computing the value of the simulation stability index based on the RDF, the simulation quantity, the temperature, and an initial quantity of atoms.

10. The method of claim 8, further comprising:
obtaining a first simulation output at a first timestep using the simulation tool,
computing the value of the simulation stability index based on the first simulation output; generating, using the trained MLFF model, an atomic force state based on the first simulation output; and
generating a second simulation output based on the atomic force state.

11. A non-transitory computer-readable storage medium storing one or more programs comprising instructions that, when executed by a processor, cause the processor to perform operations comprising:
obtaining, using the MLFF model, edge features corresponding to a training sample, wherein the training sample includes data related to a plurality of atoms, and the edge features represent relationship between edges of each atom among the plurality of atoms;
computing a correlation loss corresponding to the edge features, wherein the correlation loss represents correlation between edge features corresponding to the plurality of atoms;
updating parameters of the MLFF model based on the correlation loss to obtain a trained MLFF model; and
generating, using the trained MLFF model, a molecular dynamics (MD) simulation based on an input sample.

12. An apparatus for training a machine learning force fields (MLFF) model, the apparatus comprising:
at least one processor;
at least one memory storing instructions executable by the at least one processor;
an edge feature module comprising parameters stored in the at least one memory and configured to obtain, using the MLFF model, edge features corresponding to a training sample, wherein the training sample includes data related to a plurality of atoms, and the edge features represent relationship between edges of each atom among the plurality of atoms;
a correlation loss module comprising parameters stored in the at least one memory and configured to compute a correlation loss corresponding to the edge features, wherein the correlation loss represents correlation between edge features corresponding to the plurality of atoms; and
an updating module comprising parameters stored in the at least one memory and configured to update parameters of the MLFF model based on the correlation loss to obtain a trained MLFF model.

13. The apparatus of claim 12, wherein:
the correlation loss module is further configured to compute a total training loss based on the correlation loss, a force loss corresponding to force of the plurality of atoms, and an energy loss corresponding to energy of the plurality of atoms, and update the parameters of the MLFF model based on the total training loss.

14. The apparatus of claim 13, wherein:
the updating module is configured to determine a weight corresponding to the correlation loss in a first training timestep, and compute a weighted correlation loss based on the first weight and the correlation loss, wherein the total training loss is computed based on the weighted correlation loss.

15. The apparatus of claim 14, wherein:
the updating module is configured to determine a second weight corresponding to the correlation loss based on a second training timestep, a predetermined initial weight for a correlation loss in the second training timestep, and a predetermined update interval for a correlation loss in the second training timestep.

16. The apparatus of claim 13, further comprising:
a prediction module configured to generate predicted force and predicted energy of the plurality of atoms, compute the force loss based on the predicted force and real force of each of the plurality of atoms, and compute the energy loss corresponding based on the predicted energy and real energy of each of the plurality of atoms.

17. The apparatus of claim 12, wherein:
the edge feature module is configured to generate an edge feature matrix in each layer of a plurality of layers of the MLFF model based on the training sample
the correlation value module is configured to compute a correlation value based on the edge feature matrix, compute a correlation loss corresponding to the edge feature matrix at each layer based on the correlation value, and
the updating module is configured to update parameters of the MLFF model based on the correlation loss.

18. The apparatus of claim 17, wherein:
the correlation loss module is configured generate a correlation matrix corresponding to the edge feature matrix at each layer based on the correlation value, wherein the correlation loss is computed based on the correlation matrix c and a predetermined diagonal matrix.

19. The apparatus of claim 12, further comprising:
a simulation output module is configured to obtain a simulation output using the trained MLFF model;
a simulation stability index module is configured to compute a value of a simulation stability index of the MLFF model based on the simulation output; and
an output module is configured to display the value of the simulation stability index.

20. The apparatus of claim 19, wherein:
the simulation output module is configured to computing a radial distribution function (RDF) value of each atom pair of the plurality of atom based on the simulation positions and a simulation quantity of the plurality of atoms.
